(19) United States
(12) Patent Application Publication
(10) Pub. No.: US 2025/0041360 A1
(43) Pub. Date: Feb. 6, 2025

(54) NOVEL BIFIDOBACTERIUM BIFIDUM HEM972 STRAIN, AND COMPOSITION FOR IMPROVING INTESTINAL ENVIRONMENT, COMPRISING SAME STRAIN OR CULTURE OF SAME

(71) Applicant: HEM PHARMA INC., Pohang-si (KR)

(72) Inventors: So Young PARK, Suwon-si (KR); Yo Sep JI, Suwon-si (KR)

(21) Appl. No.: 18/919,581

(22) Filed: Oct. 18, 2024

Related U.S. Application Data
(63) Continuation of application No. PCT/KR2023/004281, filed on Mar. 30, 2023.

(30) Foreign Application Priority Data
Apr. 28, 2022 (KR) 10-2022-0052727

(51) Int. Cl. A61K 35/745; A61P 1/00

(52) U.S. Cl. CPC A61K 35/745; A61P 1/00

(57) ABSTRACT

The present disclosure relates to a strain Bifidobacterium bifidum HEM972 (KCTC14144BP), a composition for improving the intestinal environment, comprising the strain, and a composition for treating or preventing intestinal diseases, comprising the strain. A strain Bifidobacterium bifidum HEM972 (KCTC14144BP) according to an embodiment of the present disclosure can promote the production of beneficial short chain fatty acids, such as butyrate and propionate, and inhibit the production of harmful short chain fatty acids, such as isobutyrate, and thus can improve an intestinal environment or treat or prevent intestinal diseases. Therefore, the strain can be applied to pharmaceutical compositions, food compositions, health functional food compositions, feed compositions, etc. Under the provisions of the Budapest Treaty on the International Recognition of the Deposit of Microorganisms for the Purposes of Patent Procedure, the BIFIDOBACTERIUM BIFIDUM HEM972 strain was deposited with the international depositary authority: the Korean Collection for Type Cultures on Feb. 21, 2020, under the Accession Number: KCTC 14144BP.

### TECHNICAL FIELD

The present disclosure relates to a strain Bifidobacterium bifidum HEM972 (KCTC14144BP), a composition for improving an intestinal environment, comprising the strain, and a composition for treating or preventing intestinal diseases, comprising the strain.

### BACKGROUND

The modern diet, which is heavy in meat, instant and processed foods, and excessive intake of stimulating foods, along with rushed eating patterns due to a busy lifestyle, leads to undesirable eating habits and unhealthy lifestyles. As people age, harmful bacteria tend to outnumber beneficial bacteria, disrupting the balance of intestinal flora. This imbalance adversely affects intestinal health and can lead to indigestion, constipation, and colorectal cancer. Therefore, various measures, such as exercise therapy and the intake of health functional foods, are being researched to prevent and alleviate these diseases.

Microbiota refers to a microbial community in an environment as a microbial flora, and it is known that the microbiota plays an important role in maintaining homeostasis of a host, for example, human immunity, metabolites, and the like. The microbiota and the host exchange chemical signals, and the expression of immune cells, the production of neurotransmitters, short chain fatty acids (SCFA), and the like by the microbiota have a hypertrophic effect on a host system, and particularly, probiotics/prebiotics balance the unbalanced microbiota of the host, so that the metabolites of the healthy microbiota may improve the health of the host.

As a result of research on compositions for improving an intestinal environment, there have been disclosed a composition containing water extracts of red yeast rice as active ingredients (Korean Patent No. 10-1850308) and a composition containing ethanol extracts of Codonopsis lanceolata as active ingredients (Korean Patent No. 10-1826673). However, there remains a need for the development and research of compositions that exhibit superior effects in improving the intestinal environment and treating intestinal diseases.

Therefore, the present inventors have made extensive efforts to develop an excellent composition capable of improving the intestinal environment and treating or preventing intestinal diseases. As a result, the present inventors developed a novel strain that can promote the production of beneficial short chain fatty acids and inhibit the production of harmful short chain fatty acids, and then completed the present disclosure.

### DISCLOSURE OF THE INVENTION

### Problems to be Solved by the Invention

The present disclosure is conceived to provide a strain Bifidobacterium bifidum HEM972 (KCTC14144BP), a composition for improving an intestinal environment, comprising the strain, and a composition for treating or preventing intestinal diseases, comprising the strain.

However, the problems to be solved by the present disclosure are not limited to the above-described problems. Although not described herein, other problems to be solved by the present disclosure can be clearly understood by a person with ordinary skill in the art from the following descriptions.

### Means for Solving the Problems

A first aspect of the present disclosure provides a strain Bifidobacterium bifidum HEM972 (KCTC14144BP).

A second aspect of the present disclosure provides a food composition for improving an intestinal environment, comprising a strain Bifidobacterium bifidum HEM972 (KCTC14144BP) or a cultured product thereof as an active ingredient.

A third aspect of the present disclosure provides a pharmaceutical composition for treating or preventing intestinal diseases, comprising a strain Bifidobacterium bifidum HEM972 (KCTC14144BP) or a cultured product thereof as an active ingredient.

### Effects of the Invention

A strain Bifidobacterium bifidum HEM972 (KCTC14144BP) according to an embodiment of the present disclosure can promote the production of beneficial short chain fatty acids, such as butyrate and propionate, and inhibit the production of harmful short chain fatty acids, such as isobutyrate, and thus can improve an intestinal environment or treat or prevent intestinal diseases. Therefore, the strain can be applied to pharmaceutical compositions, food compositions, health functional food compositions, feed compositions, etc.

### BRIEF DESCRIPTION OF THE DRAWINGS

FIG. 1 is a diagram schematically illustrating a method for measuring the ability of a novel strain to promote and inhibit the production of short chain fatty acids.

FIG. 2 shows the result of confirming the ability of the novel strain to promote the production of butyrate.

FIG. 3 shows the result of confirming the ability of the novel strain to promote the production of propionate.

FIG. 4 shows the result of confirming the ability of the novel strain to inhibit the production of isobutyrate.

FIG. 5 is the result showing that the novel strain does not produce short chain fatty acids by itself.

### BEST MODE FOR CARRYING OUT THE INVENTION

Hereafter, examples will be described in detail with reference to the accompanying drawings so that the present disclosure may be readily implemented by a person with ordinary skill in the art. However, it is to be noted that the present disclosure is not limited to the examples but can be embodied in various other ways. In the drawings, parts irrelevant to the description are omitted for the simplicity of explanation, and like reference numerals denote like parts through the whole document.

Through the whole document, the term “on” that is used to designate a position of one component with respect to another component includes both a case that the one component is adjacent to the other component and a case that any other component exists between these two components.

Further, through the whole document, the term “comprises or includes” and/or “comprising or including” used in the document means that one or more other components, steps, operation and/or existence or addition of elements are not excluded in addition to the described components, steps, operation and/or elements unless context dictates otherwise. Through the whole document, the term “about or approximately” or “substantially” is intended to have meanings close to numerical values or ranges specified with an allowable error and intended to prevent accurate or absolute numerical values disclosed for understanding of the present disclosure from being illegally or unfairly used by any unconscionable third party. Through the whole document, the term “step of” does not mean “step for”.

Through the whole document, the term “combination(s) of” included in Markush type description means mixture or combination of one or more components, steps, operations and/or elements selected from a group consisting of components, steps, operation and/or elements described in Markush type and thereby means that the disclosure includes one or more components, steps, operations and/or elements selected from the Markush group.

Through the whole document, a phrase in the form “A and/or B” means “A or B, or A and B”.

Hereafter, embodiments and examples of the present disclosure will be described in detail with reference to the accompanying drawings. However, the present disclosure may not be limited to the following embodiments, examples, and drawings.

A first aspect of the present disclosure provides a strain Bifidobacterium bifidum HEM972 (KCTC14144BP).

In an embodiment of the present disclosure, the strain may promote the production of beneficial short chain fatty acids. Specifically, the strain may increase a content ratio of beneficial short chain fatty acids in all the short chain fatty acids.

In an embodiment of the present disclosure, the strain may promote the production of butyrate. Specifically, the strain may promote the production of butyrate in an intestinal environment or microbiota.

Through the whole document, the term “butyrate (butyric acid)” is a beneficial metabolite produced by the fermentation of carbohydrates by intestinal flora and is used as an energy source for colorectal cells. It also inhibits colorectal inflammation and enhances the function of tight junctions in colorectal cells and thus keeps the colon health and plays a role in regulating immunity. Further, it is known to influence cell apoptosis, inhibit colorectal cancer and prevent various intestinal diseases.

Therefore, it can be seen that Bifidobacterium bifidum HEM972 of the present disclosure promotes the production of butyrate in the intestinal environment or microbiota and thus increases a content ratio of butyrate in the intestinal short chain fatty acids. Thus, it can inhibit colorectal inflammation, regulate colorectal immunity, and inhibit colorectal cancer.

In an embodiment of the present disclosure, the strain may promote the production of propionate. Specifically, the strain may promote the production of propionate in the intestinal environment or microbiota.

Through the whole document, the term “propionate (propionic acid)” is a normal metabolite produced by the fermentation of carbohydrates by intestinal flora and is known to be beneficial for intestinal health by having anti-inflammatory and anti-cancer effects, inhibiting the production of fat, and helping with the colorectal cell surface and irritable bowel syndrome.

Therefore, it can be seen that Bifidobacterium bifidum HEM972 of the present disclosure promotes the production of propionate in the intestinal environment or microbiota and increases the proportion of propionate in the intestinal short chain fatty acids. Thus, it exhibits anti-inflammatory, anti-cancer and fat production inhibition effects as well as improvement in colorectal cell surface and treatment or prevention of irritable bowel syndrome.

In an embodiment of the present disclosure, the strain may inhibit the production of harmful short chain fatty acids. Specifically, the strain may decrease a content ratio of harmful short chain fatty acids in all the short chain fatty acids.

In an embodiment of the present disclosure, the strain may inhibit the production of at least one selected from the group consisting of isobutyrate and isovaleric acid.

In an embodiment of the present disclosure, the strain may inhibit the production of isobutyrate.

Through the whole document, the term “isobutyrate (isobutyric acid)” is a harmful metabolite produced by abnormal fermentation of intestinal flora. It is found in higher concentrations in patients with various intestinal diseases, specifically irritable bowel syndrome, ulcerative colitis, colorectal cancer, celiac disease, Crohn's disease, Low Anterior Resection Syndrome (LARS), and Leaky Gut Syndrome. Thus, it is known to be closely related to deterioration of intestinal health.

Therefore, it can be seen that Bifidobacterium bifidum HEM972 of the present disclosure inhibits the production of isobutyrate in the intestinal environment or microbiota and decreases a content ratio of isobutyrate in the intestinal short chain fatty acids. Thus, it can treat or prevent various intestinal diseases.

In an embodiment of the present disclosure, the strain may not produce short chain fatty acids by itself. Specifically, the strain may promote the production of beneficial short chain fatty acids in the intestinal environment, intestinal substances and/or microbiota.

In an embodiment of the present disclosure, the strain may improve the intestinal environment or may treat or prevent intestinal diseases. Specifically, the strain may be contained in various compositions, such as pharmaceutical compositions, food compositions, health functional food compositions, feed compositions, and the like, for improving the intestinal environment or treating intestinal diseases.

A second aspect of the present disclosure provides a food composition for improving an intestinal environment, comprising a strain Bifidobacterium bifidum HEM972 (KCTC14144BP) or a cultured product thereof as an active ingredient. The features described above in respect of the first aspect of the present disclosure may equally apply to the food composition according to the second aspect of the present disclosure.

Through the whole document, the term “alleviate” refers to all activities improving or making better the intestinal environment by administering the composition.

Through the whole document, the term “intestinal environment improvement” refers to advantageously changing the composition of intestinal flora and metabolites of the intestinal flora, improving microbiota or improving the intestinal function and intestinal health. The intestinal environment improvement results in an increase in beneficial intestinal bacteria and metabolites of the beneficial bacteria and thus has effects, such as vitamin synthesis, improvement of digestion and absorption, prevention of infection, and immunopotentiation, and also results in a decrease in harmful bacteria and metabolites of the harmful bacteria and thus has effects, such as decrease in intestinal putrefaction, decrease in bacterial toxins and decrease in carcinogens. Also, the intestinal environment improvement can prevent or treat intestinal diseases, such as diarrhea, constipation and enteritis, and also prevent or treat cancers, obesity, diabetes, and brain-related diseases.

In an embodiment of the present disclosure, the intestinal environment improvement may include at least one selected from the group consisting of an increase in microbial diversity of microbiota, an improvement of intestinal microbiota, an enhancement of beneficial intestinal bacteria, an improvement and enhancement of intestinal health, a decrease in endotoxin and hydrogen sulfide derived from intestinal flora, an increase in metabolites derived from beneficial microbiota, an increase in beneficial short chain fatty acids, a decrease in harmful short chain fatty acids, an increase in kind and number of beneficial bacteria and a decrease in kind and number of harmful bacteria, but may not be limited thereto.

In an embodiment of the present disclosure, the composition may be a composition for improving intestinal microbiota, a composition for enhancing beneficial intestinal bacteria, or a composition for improving intestinal health.

In an embodiment of the present disclosure, the composition may promote the production of beneficial short chain fatty acids. Specifically, the composition may promote the production of butyrate and/or propionate.

In an embodiment of the present disclosure, the composition may inhibit the production of harmful short chain fatty acids. Specifically, the composition may inhibit the production of isobutyrate and/or isovaleric acid. More specifically, the composition may inhibit the production of isobutyrate.

In an embodiment of the present disclosure, the composition may promote the production of beneficial short chain fatty acids in the intestinal environment, intestinal substances and/or microbiota.

In an embodiment of the present disclosure, the composition may treat or prevent intestinal diseases.

In an embodiment of the present disclosure, the composition may contain a strain Bifidobacterium bifidum HEM972, live cells, heat-killed cells, culture fluid, fragments and/or extracts thereof.

Through the whole document, the term “heat-killed bacteria” is opposite to the term “live bacteria” and refers to bodies obtained by suppressing the growth of bacteria, such as heat-treating live bacteria obtained by fermentation and metabolites thereof. The heat-killed bacteria may contain cytoplasm, cell wall, antibacterial substances, such as bacteriocin, polysaccharides, organic acid, and the like. Products using the heat-killed bacteria have higher stability than live bacteria products and are particularly excellent in heat resistance and have high stability to the external environment. Therefore, the products using the heat-killed bacteria are easier to store and have longer shelf life than the existing live bacteria products. Further, since the regulations on the use of antibiotics become stricter, there are a handful of companies that have produced heat-killed bacteria products. Therefore, considering the application as substitutes and the number of the producing companies, the marketability and growth potential is very high.

Through the whole document, the term “culture fluid” refers to a substance obtained by culturing the strain of the present disclosure in a known liquid medium or solid medium and may be interchangeably used with “cultured product”.

Through the whole document, the term “food” may include meats, sausages, breads, chocolates, candies, snacks, cookies, pizza, ramens, other noodles, gums, dairy products including ice cream, soups, beverages, teas, drinks, alcohol drinks, vitamin complexes, health functional foods and health foods, and may include all foods in the accepted meaning.

Through the whole document, the term “health functional food” refers to foods prepared and processed using raw materials or ingredients having useful functions to the human body in accordance with the Health Functional Food Act, No. 6727, and the “functionality” refers to adjusting nutrients on a structure and a function of the human body or obtaining a useful effect for health, such as a physiological action.

The food of the present disclosure can be manufactured by conventional methods used in the art, and can be manufactured by adding conventional raw materials and ingredients used in the art. Further, a formulation of the food is not limited as long as the formulation is accepted as a food. The food composition of the present disclosure may be prepared in a variety of formulations. Since the food is used as raw materials, unlike general drugs, the food composition is free from side effects which may occur when a drug is taken for a long time, and may have excellent portability. Therefore, the food of the present disclosure may be taken as a supplement for enhancing the effects of improving the intestinal environment.

The health food refers to a food having effects of actively maintaining or promoting health conditions, as compared with general foods, and a health supplement food refers to a food for supplementing health. If necessary, the health functional food, health food and health supplement food may be interchangeably used with each other. Specifically, the health functional food is a food prepared by adding Bifidobacterium bifidum HEM972 of the present disclosure to food materials, such as beverages, teas, spices, gums, confectionery, etc., or prepared in a capsule, a powder or a suspension form. The health functional food means that it has a specific effect on health when consumed, but unlike general drugs, the health functional food is free from side effects that may occur when a drug is taken for a long time since the food is used as raw materials.

Since the food composition of the present disclosure can be routinely ingested, the food composition is expected to show a high efficacy on the improvement of intestinal environment and thus can be very usefully applied.

The food composition may further contain a physiologically acceptable carrier. The kind of the carrier is not particularly limited. Any carrier may be used as long as it is commonly used in the art.

Further, the food composition may further contain additional ingredients that are commonly used in food compositions to improve smell, taste, visuality, etc. For example, the food composition may contain vitamins A, C, D, E, B1, B2, B6, B12, niacin, biotin, folate, pantothenic acid, etc. Furthermore, the food composition may also contain minerals, such as zinc (Zn), iron (Fe), calcium (Ca), chromium (Cr), magnesium (Mg), manganese (Mn), copper (Cu), chromium (Cr), etc. Moreover, the food composition may also contain amino acids, such as lysine, tryptophane, cysteine, valine, etc.

Further, the food composition may also contain food additives, such as preservatives (potassium sorbate, sodium benzoate, salicylic acid, sodium dehydroacetate, etc.), disinfectants (bleaching powder, higher bleaching powder, sodium hypochlorite, etc.), antioxidants (butylhydroxyanisole (BHA), butylhydroxytoluene (BHT), etc.), colorants (tar color, etc.), color-developing agents (sodium nitrite, etc.), bleaching agents (sodium sulfite), seasonings (monosodium glutamate (MSG), etc.), sweeteners (dulcin, cyclemate, saccharin, sodium, etc.), flavors (vanillin, lactones, etc.), swelling agents (alum, potassium D-bitartrate, etc.), fortifiers, emulsifiers, thickeners (adhesive pastes), film-forming agents, gum base agents, antifoaming agents, solvents, improvers, etc. The additives may be selected and used in an appropriate amount depending on the type of food.

The strain Bifidobacterium bifidum HEM972 of the present disclosure may be added as it is, or may be used in conjunction with other foods or food ingredients, and may be appropriately used according to a conventional method. The mixing amount of active ingredients may be appropriately determined depending on the purpose of use (prophylactic, health or therapeutic treatment). In general, when a food or a beverage is manufactured, the food composition of the present disclosure may be added in an amount of 50 parts by weight or less, specifically 20 parts by weight or less based on the total weight of the food or the beverage. However, when taken for the purpose of health and hygiene, the food composition may be contained in an amount below the range. In addition, since there is no safety problem, the active ingredients may be used in an amount above the range.

The food composition of the present disclosure may be used as, for example, a health beverage composition, and in this case, the health beverage composition may further contain various flavors or natural carbohydrates, as in common beverages. The natural carbohydrates may include monosaccharides, such as glucose and fructose; disaccharides, such as maltose and sucrose; polysaccharides, such as dextrin and cyclodextrin; and sugar alcohols, such as xylitol, sorbitol and erythritol. The sweeteners may be natural sweetener, such as thaumatin or a stevia extract; or synthetic sweetener, such as saccharin or aspartame. The natural carbohydrate may be generally used in an amount of from about 0.01 g to about 0.04 g, and specifically, from about 0.02 g to about 0.03 g based on 100 mL of the health beverage composition of the present disclosure.

In addition, the health beverage composition may contain various nutrients, vitamins, minerals, flavors, colorants, pectic acid and salts thereof, alginic acid and salts thereof, organic acid, protective colloidal thickeners, pH regulators, stabilizers, antiseptics, glycerin, alcohols or carbonating agents. Moreover, the health beverage composition may contain fruit flesh used to prepare natural fruit juices, fruit juice beverages or vegetable beverages. These ingredients may be used individually or in combination. A proportion of the additives is not critical, but is generally selected from 0.01 part by weight to 0.1 part by weight per 100 parts by weight of the health beverage composition of the present disclosure.

The food composition of the present disclosure may contain Bifidobacterium bifidum HEM972 of the present disclosure in a variety of % by weight as long as it can exhibit the effect of preventing hair loss and promoting hair growth. Specifically, Bifidobacterium bifidum HEM972 of the present disclosure may be contained in an amount of 0.00001% by weight to 100% by weight or 0.01% by weight to 80% by weight based on the total weight of the food composition, but may not be limited thereto.

In an embodiment of the present disclosure, the food composition may be a health functional food composition.

A third aspect of the present disclosure provides a pharmaceutical composition for treating or preventing intestinal diseases, comprising a strain Bifidobacterium bifidum HEM972 (KCTC14144BP) or a cultured product thereof as an active ingredient. The features described above in respect of the first and second aspects of the present disclosure may equally apply to the pharmaceutical composition according to the third aspect of the present disclosure.

In an embodiment of the present disclosure, the composition may contain a strain Bifidobacterium bifidum HEM972, live bacterial cell bodies, heat-killed bodies, culture fluid, fragments and/or extracts thereof.

Through the whole document, the term “intestinal diseases” refer to any diseases that can arise or develop due to the deterioration of the intestinal environment, imbalance of intestinal flora (microbiota), deterioration of intestinal health, or intestinal damage. This can include intestinal damage, gastric diseases, digestive tract diseases, and digestive tract damage.

In an embodiment of the present disclosure, the intestinal diseases may include at least one selected from the group consisting of inflammatory intestinal disease, irritable bowel syndrome (IBS), ulcerative colitis (UC), colorectal cancer, celiac disease, Crohn's disease, low anterior resection syndrome (LARS), and leaky gut syndrome, but is not limited thereto as long as it can develop due to abnormal intestinal conditions.

Through the whole document, the term “treat” refers to all activities reducing or alleviating symptoms of intestinal diseases by administering a pharmaceutical composition containing Bifidobacterium bifidum HEM972 of the present disclosure as an active ingredient to a subject with an intestinal disease.

In an embodiment of the present disclosure, the composition may promote the production of beneficial short chain fatty acids. Specifically, the composition may promote the production of butyrate and/or propionate.

In an embodiment of the present disclosure, the composition may inhibit the production of harmful short chain fatty acids. Specifically, the composition may inhibit the production of isobutyrate and/or isovaleric acid. More specifically, the composition may inhibit the production of isobutyrate.

In an embodiment of the present disclosure, the composition may promote the production of beneficial short chain fatty acids in the intestinal environment, intestinal substances and/or microbiota.

In an embodiment of the present disclosure, the composition may improve the intestinal environment.

In an embodiment of the present disclosure, the pharmaceutical composition may be formulated and used in the form of oral dosage forms, such as powders, granules, tablets, capsules, suspensions, emulsions, syrups, aerosols, etc., external preparations, suppositories, or sterile injection solutions, according to a conventional method, but may not be limited thereto.

In an embodiment of the present disclosure, the pharmaceutical composition may be formulated with generally used diluents or excipients, such as fillers, bulking agents, binders, wetting agents, disintegrating agents or surfactants, but may not be limited thereto.

In an embodiment of the present disclosure, solid formulations for oral administration may include tablets, pills, powders, granules or capsules, and these solid formulations may be prepared by mixing heat-killed bacteria of the strain with at least one of excipients, such as starch, calcium carbonate, sucrose, lactose or gelatin. Except for the simple excipients, lubricants, such as magnesium stearate or talc, may be used, but the present disclosure may not be limited thereto.

In an embodiment of the present disclosure, liquid formulations for oral administration may include suspensions, solutions for internal use, emulsions and syrups, and may contain various excipients, such as wetting agents, sweeteners, aromatics and preservatives, in addition to generally used simple diluents, such as water and liquid paraffin, but may not be limited thereto.

In an embodiment of the present disclosure, formulations for parenteral administration may include sterilized aqueous solutions, water-insoluble excipients, suspensions, emulsions, lyophilized preparations and suppositories, but may not be limited thereto. For example, the water insoluble excipients or suspensions may contain propylene glycol, polyethylene glycol, vegetable oil, such as olive oil, injectable ester, such as ethylolate, and the like, but may not be limited thereto. For example, the suppositories may contain witepsol, macrogol, tween 61, cacao butter, laurin butter, glycerol, gelatin, and the like, but may not be limited thereto.

The pharmaceutical composition according to an embodiment of the present disclosure may be a drug composition or a quasi-drug composition.

Through the whole document, the term “quasi-drug” refers to an article having a milder action than drugs, among articles being used for the purpose of diagnosis, treatment, improvement, alleviation, handling or prevention of human or animal diseases. For example, according to the Pharmaceutical Affairs Law, the quasi-drugs are those, excluding articles used as drugs, including articles used for the purpose of treating or preventing human or animal diseases and articles which have a mild action on or have no direct influence on the human body.

The quasi-drug composition of the present disclosure may be manufactured in a formulation selected from the group consisting of body cleanser, sanitizer, detergent, kitchen cleanser, detergent for cleaning, toothpaste, mouthwash, wet wipe, cleanser, soap, hand soap, hair cleanser, hair softener, humidifying filler, mask, ointment or filter filler, but may not be limited thereto.

In an embodiment of the present disclosure, the pharmaceutical composition may be administered in a pharmaceutically effective amount. Through the whole document, the term “pharmaceutically effective amount” refers to an amount sufficient to treat or prevent diseases at a reasonable benefit/risk ratio applicable to any medical treatment or prevention. An effective dosage level may be determined depending on factors including severity of the disease, drug activity, a patient's age, body weight, health conditions, gender, sensitivity to the drug, administration time, administration route, and excretion rate of the composition of the present disclosure, duration of treatment, drugs blended with or co-administered with the composition of the present disclosure, and other factors known in the medical field. The pharmaceutical composition of the present disclosure may be administered individually or in combination with an ingredient known for treating intestinal diseases. It is important to administer an amount to obtain a maximum effect in a minimum amount without side effects by considering all of the above-described factors.

In an embodiment of the present disclosure, an administration dose of the pharmaceutical composition may be determined by a person with ordinary skill in the art in view of purpose of use, severity of the disease, a patient's age, body weight, gender, medical history or the kind of a material used as an active ingredient. For example, the pharmaceutical composition of the present disclosure may be administered at a dose of from about 0.1 ng/kg to about 1,000 mg/kg, and preferably, from about 1 ng/kg to about 100 mg/kg per adult, and the administration frequency of the composition of the present disclosure is not particularly limited, but the composition may be administered once a day or several times a day in divided doses. The administration dose or the administration frequency does not limit the scope of the present disclosure in any aspect.

A fourth aspect of the present disclosure provides a method for treating intestinal diseases, comprising administering the pharmaceutical composition of the present disclosure to a subject suffering from an intestinal disease in a pharmaceutically effective amount. The features described above in respect of the first to third aspects of the present disclosure may equally apply to the method according to the fourth aspect of the present disclosure.

Through the whole document, the term “subject” may include, without limitation, mammals including mice, livestock and humans, and farmed fish which develop or are at risk of developing intestinal diseases.

In an embodiment of the present disclosure, the subject may be other than a human.

In an embodiment of the present disclosure, the pharmaceutical composition may be administered singly or multiply in a pharmaceutically effective amount. In this case, the composition may be formulated and administered in the form of a solution, powder, aerosol, injection, infusion (Ringer solution), capsule, pill, tablet, suppository or patch.

The pharmaceutical composition for preventing or treating intestinal diseases of the present disclosure may be administered via any route as long as the route allows the pharmaceutical composition to reach a target tissue.

The pharmaceutical composition of the present disclosure may be administered via, but not particularly limited to, intraperitoneal administration, intravenous administration, intramuscular administration, subcutaneous administration, intradermal administration, transdermal patch administration, oral administration, intranasal administration, intrapulmonary administration, rectal administration, etc. depending on the purpose. However, when the pharmaceutical composition is administered via oral administration, it can be administered in an unformulated form, and since the strain Bifidobacterium bifidum HEM972 can be denatured or degraded by gastric acid, the composition for oral administration may be coated with an active drug, formulated to be protected from degradation in the stomach, or formulated in the form or an oral patch. Also, the composition may be administered by any device capable of delivering an active ingredient to a target cell.

A fifth aspect of the present disclosure provides a method for improving an intestinal environment, comprising administering the food composition of the present disclosure to a subject suffering from an intestinal disease.

A sixth aspect of the present disclosure provides a method for preventing intestinal diseases, comprising administering the pharmaceutical composition to a subject suffering from an intestinal disease.

### MODE FOR CARRYING OUT THE INVENTION

Hereinafter, the present disclosure will be explained in more detail with reference to Examples. However, the following Examples are illustrative only for better understanding of the present disclosure, but do not limit the present disclosure.

### EXAMPLES

### Example 1: Confirmation of Ability of Bifidobacterium bifidum HEM972 to Promote and Inhibit Production of Short Chain Fatty Acid

To verify the ability to promote the production of short chain fatty acids of a novel strain Bifidobacterium bifidum HEM972 (Depository Institution: Korean Collection for Type Cultures at Korean Research Institute of Bioscience & Biotechnology, Accession Number: KCTC14144BP and Date of Deposit: Feb. 21, 2020) of the present disclosure, a test was conducted as described below.

### Example 1-1: In Vitro Culture for Analysis of Short Chain Fatty Acid Production

In order to check the amount of short chain fatty acid produced by the strain of the present disclosure under in vitro conditions, the strain was cultured as follows.

First, an intestinal environment-like medium of the following composition was prepared, followed by substitution for 24 hours in an anaerobic chamber (Whitley A95 anaerobic workstation). Thus, the medium was brought into an anaerobic state.
Intestinal environment-like medium: NaCl 60 mM, NaHCO₃ 40 mM, KCl 10 mM, Hemin 5×10⁻⁵ g/L, mucin 0.5% (w/v), L-cysteine HCl 0.05% (w/v)

To each well of 96-well plates for test groups (HEM972, PB_B), 30 μl of the strain at a concentration of 5×10⁷ CFU/ml, 180 μl of an intestinal environment-like medium, and 15 mg of a human fecal sample were dispensed, and to each well for a control group NC (Negative Control), 210 μl of an intestinal environment-like medium and 15 mg of a human fecal sample were dispensed. Also, the same-genus strain Bifidobacterium, which has already been commercialized, was used for PB_B to serve as a reference. The strain HEM972 for the test groups refers to Bifidobacterium bifidum HEM972.

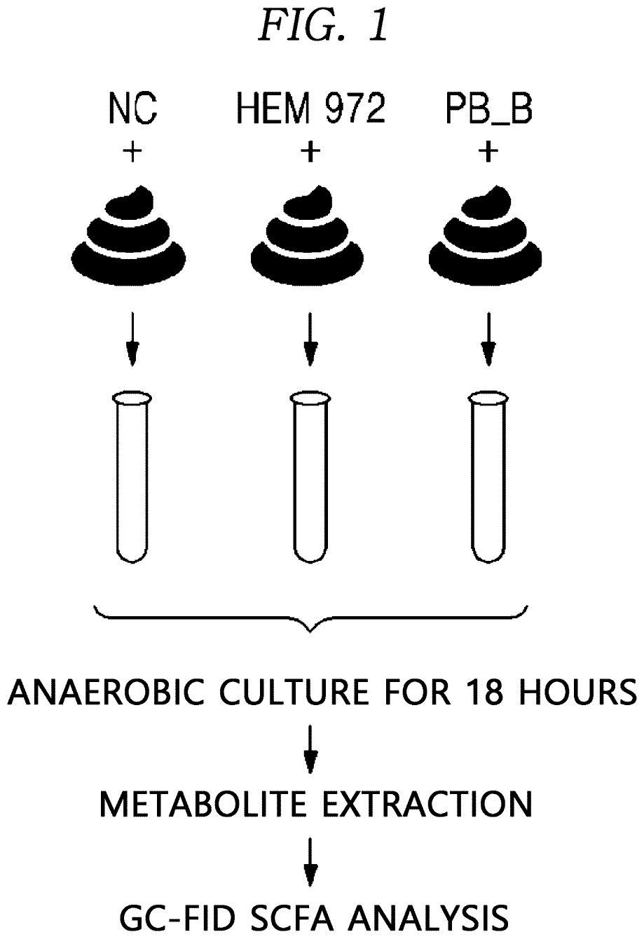

Then, the 96-well-plates for the test group and the control group were cultured with stirring using a stirrer (DLAB mx-m) in an anaerobic chamber (Whitley A95 anaerobic workstation) for 18 hours, followed by centrifugation at 3800 rpm for 10 minutes to transfer 100 μl of supernatant of each well to a 96-well cell culture plate. Thereafter, 100 μl of a GC (gas chromatography) extraction solution [GC extraction solution composition: 10 ml of DW, 3.3 g of NaCl, 50 μl of phosphoric acid and 0.5 μl of 2-ethyl butyric acid] for metabolite extraction, 50 μl of DW and 50 μl of the supernatant of the 96-well cell culture plate were dispensed to each of 96 GC vials. Then, acetate, propionate, butyrate and isobutyrate, which are microbial metabolites, were assayed by gas chromatography (FIG. 1).

### Example 1-2: Confirmation of Ability to Promote Butyrate Production

Ratio changes of butyrate as a result of treatment with the novel strain were checked by gas chromatography as performed in Example 1-1. Specifically, the ratio of butyrate can be checked from the ratio of butyrate to the total amount of normal short chain fatty acids, such as acetate, propionate, butyrate, isobutyrate, valeric acid and isovaleric acid, present in a sample.

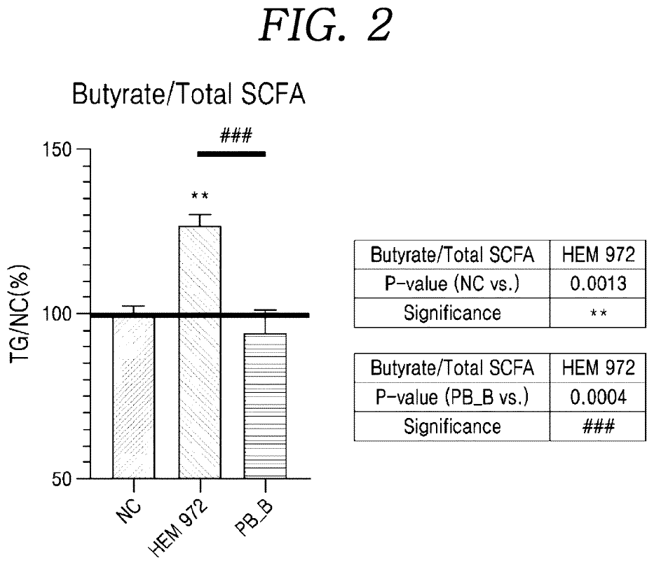

In FIG. 2, the term “Total SCFA” refers to the total amount of acetate, propionate, butyrate, isobutyrate, valeric acid and isovaleric acid, the term “Butyrate” represents the amount of butyrate, and the term “Butyrate/Total SCFA” is the ratio of butyrate obtained by dividing the amount of butyrate by “Total SCFA”. To easily check an increase or decrease of butyrate in each test group treated with the strain as compared to the untreated control group NC, Butyrate/Total SCFA of the NC is set to 100% and Butyrate/Total SCFA of each test group TG is expressed as TG/NC obtained by dividing it by NC value. Also, to compare the Butyrate/Total SCFA values of the respective test groups with the control group NC, statistical significance was checked through one-way ANOVA (post-hoc test: Fisher's LSD) of NC and HEM972 and the result was marked on the bar graph with * (P-value<0.05), ** (P-value<0.01), and *** (P-value<0.001). To compare the Butyrate/Total SCFA values of HEM972 and PB_B as a reference strain, statistical significance was checked through one-way ANOVA (post-hoc test: Fisher's LSD) and the result was marked with #(P-value<0.05), ##(P-value<0.01), and ###(P-value<0.001).

As a result of checking the increase or decrease of butyrate, it was confirmed that the ratio of butyrate was statistically significantly increased in the group treated with the strain HEM972 compared to the control group and PB_B (FIG. 2).

Based on the above result, it was found that the strain HEM972 of the present disclosure has the function of promoting the production of butyrate in the intestinal environment (microbiota).

### Example 1-3: Confirmation of Ability to Promote Propionate Production

Ratio changes of propionate as a result of treatment with the novel strain were checked by gas chromatography as performed in Example 1-1. Specifically, the ratio of propionate can be checked from the ratio of propionate to the total amount of normal short chain fatty acids, such as acetate, propionate, butyrate, isobutyrate, valeric acid and isovaleric acid, present in a sample.

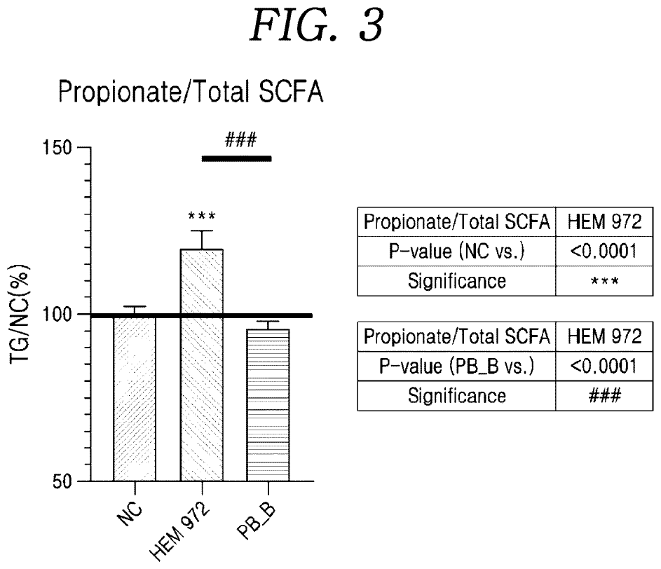

In FIG. 3, the term “Total SCFA” refers to the total amount of acetate, propionate, butyrate, isobutyrate, valeric acid and isovaleric acid, the term “Propionate” represents the amount of propionate, and the term “Propionate/Total SCFA” is the ratio of propionate obtained by dividing the amount of propionate by “Total SCFA”. To easily check an increase or decrease of propionate in each test group treated with the strain as compared to the untreated control group NC, Propionate/Total SCFA of the NC is set to 100% and Propionate/Total SCFA of each test group TG is expressed as TG/NC obtained by dividing it by NC value. Also, to compare the Propionate/Total SCFA values of the respective test groups with the control group NC, statistical significance was checked through one-way ANOVA (post-hoc test: Fisher's LSD) of NC and HEM972 and the result was marked on the bar graph with * (P-value<0.05), ** (P-value<0.01), and *** (P-value<0.001). To compare the Propionate/Total SCFA values of HEM972 and PB_B as a reference strain, statistical significance was checked through one-way ANOVA (post-hoc test: Fisher's LSD) and the result was marked with #(P-value<0.05), ##(P-value<0.01), and ###(P-value<0.001).

As a result of checking the increase or decrease of propionate, it was confirmed that the ratio of propionate was statistically significantly increased in the group treated with the strain HEM972 compared to the control group and PB_B (FIG. 3).

Based on the above result, it was found that the strain HEM972 of the present disclosure has the function of promoting the production of propionate in the intestinal environment (microbiota).

### Example 1-4: Confirmation of Ability to Inhibit Isobutyrate Production

Ratio changes of isobutyrate as a result of treatment with the novel strain were checked by gas chromatography as performed in Example 1-1.

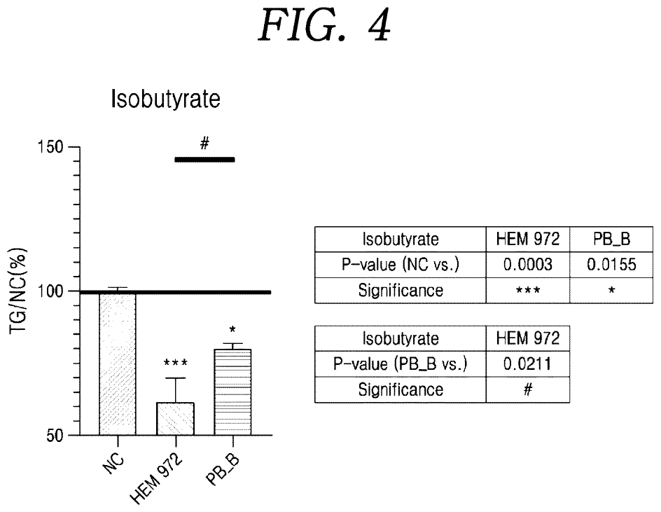

In FIG. 4, the term “Isobutyrate” represents the amount of isobutyrate, and to easily check an increase or decrease of isobutyrate in each test group treated with the strain as compared to the untreated control group NC, Isobutyrate of the NC is set to 100% and Isobutyrate of each test group TG is expressed as TG/NC obtained by dividing it by NC value. Also, to compare the Isobutyrate values of HEM972 with the control group NC, statistical significance was checked through one-way ANOVA (post-hoc test: Fisher's LSD) of NC and the test group and the result was marked on the bar graph with * (P-value<0.05), ** (P-value<0.01), and *** (P-value<0.001). To compare the Isobutyrate values of HEM972 and PB_B as a reference strain, statistical significance was checked through one-way ANOVA (post-hoc test: Fisher's LSD) and the result was marked with #(P-value<0.05), ##(P-value<0.01), and ###(P-value<0.001).

As a result of checking the increase or decrease of isobutyrate, it was confirmed that the amount of isobutyrate was statistically significantly increased in the group treated with the strain HEM972 compared to the control group and PB_B (FIG. 4).

Based on the above result, it was found that the strain HEM972 of the present disclosure has the function of inhibiting the production of isobutyrate in the intestinal environment (microbiota).

### Example 2: Confirmation of Ability of Bifidobacterium bifidum HEM972 to Produce Short Chain Fatty Acid by Itself

In Example 1, it was confirmed that the novel strain of the present disclosure, Bifidobacterium bifidum HEM972, increases the ratio of butyrate and propionate while inhibiting the production of isobutyrate. However, these tests did not clearly indicate whether the strain produces butyrate and propionate by itself or promotes the production of these short chain fatty acids in the intestinal environment. To clarify this point, the following tests were conducted.

First, an intestinal environment-like medium of the following composition was prepared, followed by substitution for 24 hours in an anaerobic chamber (Whitley A95 anaerobic workstation). Thus, the medium was brought into an anaerobic state.
Intestinal environment-like medium: NaCl 60 mM, NaHCO₃ 40 mM, KCl 10 mM, Hemin 5×10⁻⁵ g/L, mucin 0.5% (w/v), L-cysteine HCl 0.05% (w/v)

Then, to a 96-well plate for a test group A (treatment group: HEM972), 30 μl of the strain at a concentration of 5×10⁷ CFU/ml and 195 μl of an intestinal environment-like medium were dispensed, and to a 96-well plate for a test group B (treatment group: HEM972), 30 μl of the strain at a concentration of 5×10⁷ CFU/ml, 180 μl of an intestinal environment-like medium and 15 mg of a human fecal sample were dispensed. Also, to each well for an untreated control group NC (Negative Control), 225 μl of an intestinal environment-like medium was dispensed.

Then, the 96-well-plates for the test groups were cultured with stirring using a stirrer (DLAB mx-m) in an anaerobic chamber (Whitley A95 anaerobic workstation) for 18 hours, followed by centrifugation at 3800 rpm for 10 minutes to transfer 100 μl of supernatant of each well to a 96-well cell culture plate. Thereafter, 100 μl of a GC (gas chromatography) extraction solution [GC extraction solution composition: 10 ml of DW, 3.3 g of NaCl, 50 μl of phosphoric acid and 0.5 μl of 2-ethyl butyric acid] for metabolite extraction, 50 μl of DW and 50 μl of the supernatant of the 96-well cell culture plate were dispensed to each of 96 GC vials. Then, the amounts of acetate, propionate, butyrate and isobutyrate, which are microbial metabolites, as well as 2-ethyl butyric acid, which is an internal standard for GC, were assayed by gas chromatography.

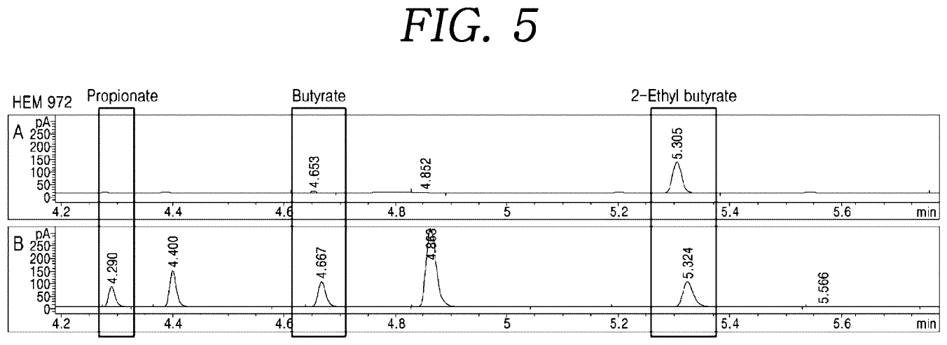

The assay result confirmed that the test group A which was treated with HEM972 only but not treated with the fecal sample did not produce butyrate and propionate, whereas the test group treated with the strain and the fecal sample produced butyrate and propionate (FIG. 5).

Based on the above result, it was found that the strain HEM972 of the present disclosure does not have the ability to produce butyrate and propionate by itself, but the strain HEM972 has the function of promoting the production of butyrate and propionate by stimulating and regulating the microbiota (fecal sample).

The above description of the present disclosure is provided for the purpose of illustration, and it would be understood by a person with ordinary skill in the art that various changes and modifications may be made without changing technical conception and essential features of the present disclosure. Thus, it is clear that the above-described examples are illustrative in all aspects and do not limit the present disclosure. For example, each component described to be of a single type can be implemented in a distributed manner. Likewise, components described to be distributed can be implemented in a combined manner.

The scope of the present disclosure is defined by the following claims rather than by the detailed description. It shall be understood that all modifications and embodiments conceived from the meaning and scope of the claims and their equivalents are included in the scope of the present disclosure.

### ACCESSION NUMBER

Depository Institution: Korean Research Institute of Bioscience & Biotechnology
Accession Number: KCTC14144BP
Date of Deposit: Feb. 21, 2020

We claim:

1. A composition comprising a strain Bifidobacterium bifidum HEM972 (KCTC14144BP).

2. The composition of claim 1,
wherein the strain promotes the production of at least one selected from the group consisting of butyrate and propionate.

3. The composition of claim 1,
wherein the strain inhibits the production of isobutyrate.

4. The composition of claim 1,
wherein the strain does not produce short chain fatty acids by itself.

5. The composition of claim 1,
wherein the composition comprises a strain Bifidobacterium bifidum HEM972, live cells, heat-killed cells, culture fluid, fragments, or extracts thereof.

6. A food composition for improving an intestinal environment, comprising:
a strain Bifidobacterium bifidum HEM972 (KCTC14144BP) or a cultured product thereof as an active ingredient.

7. A pharmaceutical composition for treating or preventing intestinal diseases, comprising:
a strain Bifidobacterium bifidum HEM972 (KCTC14144BP) or a cultured product thereof as an active ingredient.

8. A method for improving an intestinal environment, comprising:
administering to a subject in need thereof a composition of claim 1.

9. A method for treating or preventing intestinal diseases, comprising:
administering to a subject in need thereof a composition of claim 1.
